(19) United States
(12) Patent Application Publication
(10) Pub. No.: US 2026/0215806 A1
(43) Pub. Date: Jul. 30, 2026

(54) DEBRIDEMENT DEVICE FOR HUMAN DISEASED TISSUE

(71) Applicant: ZHEJIANG UNIVERSITY, Zhejiang (CN)

(72) Inventors: Hong YU, Zhejiang (CN); Bo SHEN, Zhejiang (CN); Xin YU, Zhejiang (CN); Hongyuan LV, Zhejiang (CN)

(73) Assignee: ZHEJIANG UNIVERSITY, Zhejiang (CN)

(21) Appl. No.: 18/722,497

(22) PCT Filed: Oct. 30, 2023
(86) PCT No.: PCT/CN2023/127657

§ 371 Date: Jun. 20, 2024

(30) Foreign Application Priority Data
Jun. 13, 2023 (CN) 202310701685.1

(51) Int. Cl. A61B 17/3207; A61B 17/32

(52) U.S. Cl. CPC A61B 17/320758; A61B 2017/320052; A61B 2217/005; A61B 2217/007

(57) ABSTRACT

A debridement device for human diseased tissue is provided, including an insertion tube, a rotary cutting head and an operating handle. The insertion tube includes an inner tube and an outer tube sleeved outside the inner tube, a dirt suction chamber is formed in the inner tube, and an irrigation chamber is formed between the inner tube and the outer tube. The rotary cutting head includes a housing and a rotary cutter head arranged in the housing. An interior of the housing communicates with the dirt suction chamber, a rotary cutting opening for exposing the rotary cutter head is formed in the housing, and a notch corresponding to the rotary cutting opening is formed in the outer tube. The operating handle is connected with the insertion tube and the rotary cutting head.

### CROSS REFERENCE TO RELATED APPLICATION

This application is a national stage application of International Patent Application No. PCT/CN2023/127657, filed on Oct. 30, 2023, which claims the priority of Chinese Patent Application No. 202310701685.1 entitled “DEBRIDEMENT DEVICE FOR HUMAN DISEASED TISSUE” filed with the Chinese Patent Office on Jun. 13, 2023, which is incorporated herein by reference in its entirety.

### TECHNICAL FIELD

The present disclosure relates to the field of medical devices, in particular to the field of interventional medical devices, specifically a debridement device for human diseased tissue.

### BACKGROUND

Acute pancreatitis (AP) is a common disease in digestive system. Most patients have mild symptoms and are self-limited. However, 15%-25% of AP patients will develop into severe acute pancreatitis (SAP), accompanied by pancreatic or peripancreatic tissue necrosis and systemic multiple organ failure, with a mortality rate as high as 10%-30%. The death of patients with severe pancreatitis is largely due to peripancreatic necrosis with infection that is difficult to remove during the necrotic infection phase, and thus timely and effective removal of necrotic infection of pancreas and peripancreatic tissue is a key factor related to the curative effect. In recent years, with the development of minimally invasive technology, open surgery with serious injury can be avoided for some patients, and minimally invasive surgery such as percutaneous catheter drainage (PCD), minimal access retroperitoneal pancreatic necrosectomy (MARPN) and laparoscopic-assisted drainage are chosen to reduce tissue damage and complications. At present, the minimally invasive treatment scheme adopted in clinic is mainly carried out with the assistance of laparoscopy, the abscess cavity is located by fine needle puncture, the necrotic fluid initially is drained by percutaneous puncture drainage, the abscess cavity is opened, and the necrotic tissue and pus are removed with long foreign body forceps and aspirator.

However, because the necrotic tissue of acute pancreatitis is fragile and difficult to clamp, and the pus is sticky and easy to block the aspirator, the debridement effect is often poor, the residual necrotic tissue cannot be completely removed, it is inevitable to have the second or even more operations, which increases the painful experience of the patient, affects the treatment effect of the patient, prolongs the hospitalization time of the patient, and causes heavy financial burden. Meanwhile, in the process of forceps retrieval and suction of the necrotic tissue, there is the possibility of injuring great vessels and causing massive bleeding. Therefore, there is an urgent need for a safe and efficient surgical instrument to remove necrotic infected tissue of acute pancreatitis.

### SUMMARY

A debridement device for human diseased tissue is provided by the present disclosure. The device can achieve the rotary cutting and removing of the human diseased tissue at the same time, reduce the residual necrotic substances, effectively protect the surrounding normal tissues and blood vessels, reduce the possibility that a patient needs repeated operations, and improve the efficiency, effect and safety of minimally invasive treatment.

A debridement device for human diseased tissue is provided by the present disclosure, including an insertion tube, a rotary cutting head and an operating handle. The insertion tube includes an inner tube, and an outer tube sleeved outside the inner tube, a dirt suction chamber is formed in the inner tube, and an irrigation chamber is formed between the inner tube and the outer tube. The rotary cutting head includes a housing, and a rotary cutter head arranged in the housing. An interior of the housing communicates with the dirt suction chamber, a rotary cutting opening for exposing the rotary cutter head is formed in the housing, and a notch corresponding to the rotary cutting opening is formed in the outer tube. The operating handle is connected with the insertion tube and the rotary cutting head to control the rotary cutter head to cut human tissue at the rotary cutting opening, to suck dirt from the dirt suction chamber, and to irrigate the rotary cutting head through the irrigation chamber.

Further, the rotary cutting opening is laterally formed in the housing, and a top of the housing is a blind end and is arc-shaped.

Further, the operating handle is provided with a negative pressure suction port. One end, away from the rotary cutting head, of the dirt suction chamber communicates with the negative pressure suction port, and a suction bottle and an aspirator form a negative pressure in the dirt suction chamber through the negative pressure suction port.

Further, the rotary cutter head is rotatably arranged in the housing about an axis of the rotary cutter head.

Further, the rotary cutting head includes a rotating shaft. One end of the rotating shaft is connected with the rotary cutter head, and an other end of the rotating shaft is connected with a driving component in the operating handle. The driving component drives the rotary cutter head to rotate through the rotating shaft.

Further, partitions are arranged between the inner tube and the outer tube to divide the irrigation chamber into a first irrigation channel and a second irrigation channel. An opening of one end, away from the operating handle, of the first irrigation channel is formed in one end, adjacent to the insertion tube, of the rotary cutting head, and is located on the housing at a position where the rotary cutting opening is located on the housing. One end, away from the operating handle, of the outer tube is not closed with respect to the housing, and the second irrigation channel is formed between the outer tube and a side wall of the housing at the rotary cutting head. An opening of the second irrigation channel is formed in an end portion of one end, away from the operating handle, of the housing.

Further, two end portions of the outer tube are respectively flush with end portions of the housing where the rotary cutting opening is not provided.

Further, the inner tube is provided with multiple connecting holes for communicating the dirt suction chamber with the irrigation chamber, and a flow direction control structure is arranged at the connecting holes to allow one-way flow of an irrigation solution from the irrigation chamber in one direction into the dirt suction chamber.

Further, the insertion tube is provided with a bent part, and a guide wire connected with the bent part, the bent part is arranged at a position, adjacent to the rotary cutting head, of the insertion tube, the bent part is connected with a guide wire control structure on the operating handle through the guide wire. A length of the guide wire is controlled through the operating handle, so as to control the degree of bending of the bent part.

Further, the guide wire control structure includes a rotating gear. The guide wire is wound on the rotating gear, and a fixing structure for fixing a rotating angle of the rotating gear is arranged on the rotating gear.

In conclusion, when debriding the human diseased tissue, the debridement device for human diseased tissue can be extended to a position of the diseased tissue through the incision of laparoscopic surgery, a position of the rotary cutting opening is enabled to correspond to the diseased tissue, and the rotary cutting head is controlled by the operating handle to cut the human diseased tissue. During cutting, the rotary cutting opening and surrounding areas are irrigated through the irrigation chamber, and the cut diseased tissue, irrigated sewage and other dirt are sucked out through the dirt suction chamber, so as to complete the debridement of the human diseased tissue. The device can achieve the rotary cutting and removing of the human diseased tissue at the same time, reduce the residual necrotic substances, effectively protect the surrounding normal tissues and blood vessels, reduce the possibility that a patient needs repeated operations, and improve the efficiency, effect and safety of minimally invasive treatment.

The above description is only an overview of the technical solution of the present disclosure, which can be implemented according to the contents of the specification in order to understand the technical means of the present disclosure more clearly. In order to make the above and other objectives, features and advantages of the present disclosure more obvious and understandable, the following is a detailed description of preferred embodiments with the drawings.

### BRIEF DESCRIPTION OF THE DRAWINGS

FIG. 1 is a structural schematic diagram of a debridement device for human diseased tissue according to an embodiment of the present disclosure;

FIG. 2 is a structural schematic diagram of a rotary cutting head in FIG. 1;

FIG. 3 is a structural schematic diagram of a housing of the rotary cutting head in FIG. 2;

FIG. 4 is a structural schematic diagram of a rotary cutter head at the rotary cutting head in FIG. 2;

FIG. 5 is a structural schematic diagram after the rotary cutting head and an insertion tube in FIG. 1 are combined;

FIG. 6 is a structural schematic diagram of a first irrigation channel in FIG. 5;

FIG. 7 is a structural schematic diagram of a second irrigation channel in FIG. 5;

FIG. 8 is a schematic diagram of a sectional structure at a joint of an operating handle and an insertion tube.

### DETAILED DESCRIPTION OF THE EMBODIMENTS

In order to further illustrate the technical means and efficacy adopted by the present disclosure to achieve the intended purpose, the detailed description with reference to the drawings and preferred embodiments is as follows.

A debridement device for human diseased tissue is provided by the present disclosure. The device can achieve the rotary cutting and removing of the human diseased tissue at the same time, reduce the residual necrotic substances, effectively protect the surrounding normal tissues and blood vessels, reduce the possibility that a patient needs repeated operations, and improve the efficiency, effect and safety of minimally invasive treatment.

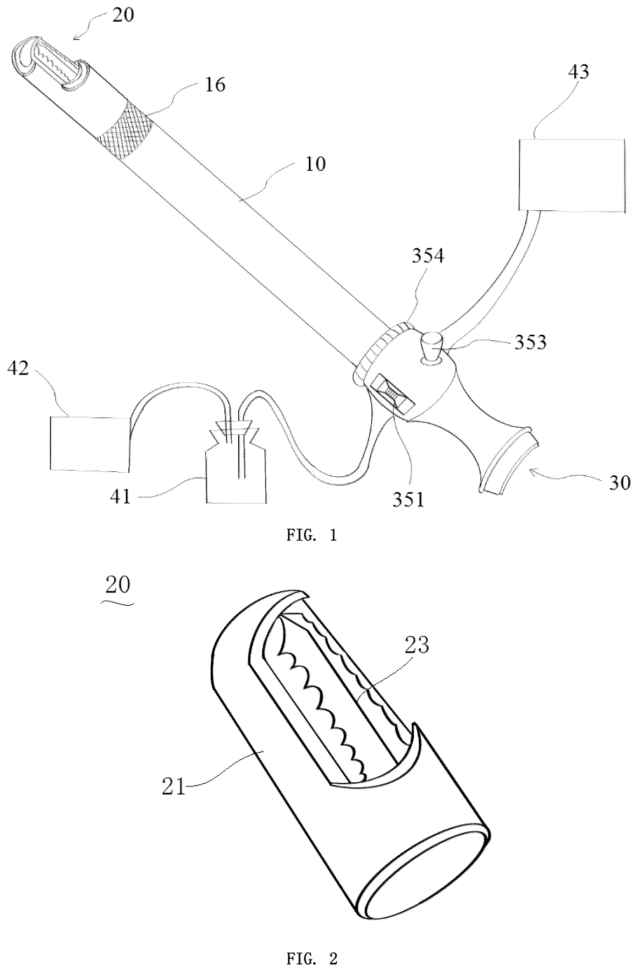

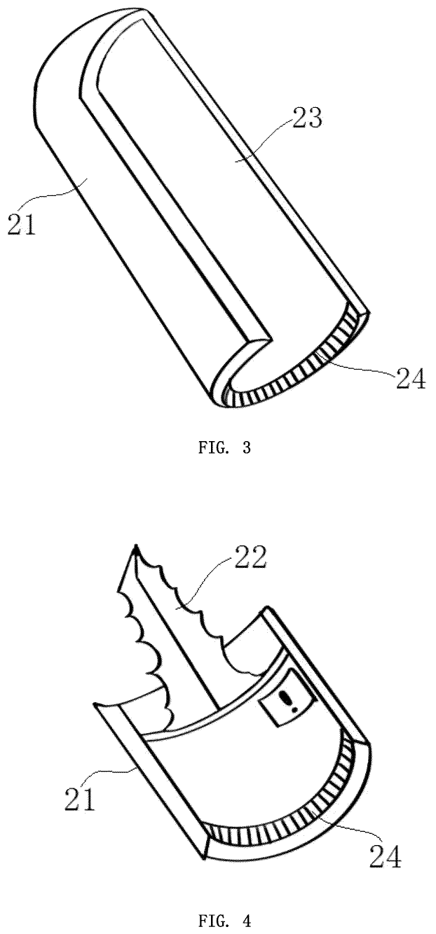

FIG. 1 is a structural schematic diagram of a debridement device for human diseased tissue provided by an embodiment of the present disclosure. FIG. 2 is a structural schematic diagram of a rotary cutting head in FIG. 1. FIG. 3 is a structural schematic diagram of a housing of the rotary cutting head in FIG. 2. FIG. 4 is a structural schematic diagram of a rotary cutter head at the rotary cutting head in FIG. 2 (for the convenience of display, a portion of the housing structure is omitted in FIG. 4). As shown in FIG. 1 to FIG. 4, the debridement device for human diseased tissue provided by the embodiment of the present disclosure includes an insertion tube 10, a rotary cutting head 20 and an operating handle 30. An insertion tube 10 includes an inner tube 11 and an outer tube 20 sleeved outside the inner tube 11, a dirt suction chamber 13 is formed in the inner tube 11, and an irrigation chamber 14 is formed between the inner tube 11 and the outer tube 12. The rotary cutting head includes a housing 21 and a rotary cutter head 22 arranged in the housing 21. The interior of the housing 21 communicates with the dirt suction chamber 13, a rotary cutting opening 23 for exposing the rotary cutter head 22 is formed in the housing 21, and a notch corresponding to the rotary cutting opening 23 is formed in the outer tube 12, such that the rotary cutter head 22 can make direct contact with the human tissue. The operating handle 30 is connected with the insertion tube 10 and the rotary cutting head 20, so as to control the rotary cutter head 22 to cut human tissue at the rotary cutting opening 23, to suck dirt from the dirt suction chamber 13, and to irrigate the rotary cutting head 20 through the irrigation chamber 14.

In this embodiment, when debriding the human diseased tissue, the debridement device for human diseased tissue can be extended to a position of the diseased tissue through the incision of laparoscopic surgery, a position of the rotary cutting opening 23 is enabled to correspond to the diseased tissue, and the rotary cutting head 20 is controlled by the operating handle 30 to cut the human diseased tissue. During cutting, the rotary cutting opening 23 and surrounding areas are irrigated through the irrigation chamber 14, and the cut diseased tissue, irrigated sewage and other dirt are sucked out through the dirt suction chamber 13, so as to complete the debridement of the human diseased tissue. The device can achieve the rotary cutting and removing of the human diseased tissue at the same time, reduce the residual necrotic substances, effectively protect the surrounding normal tissues and blood vessels, reduce the possibility that a patient needs repeated operations, and improve the efficiency, effect and safety of minimally invasive treatment.

Further, in this embodiment, the rotary cutting opening 23 is laterally formed in the housing 21, an end portion of the housing 21 of the rotary cutting head 20 is a blind end, a top end of which is in arc transition. Through the above arrangement, the rotary cutter head 22 can be accommodated into the housing 21 and make contact with the diseased tissue only through the rotary cutting opening 23, which can protect normal tissue and blood vessels during the operation, and can prevent the rotary cutter head 22 from accidentally injuring human tissue at other sites.

The rotary cutting opening 23 may be chamfered rectangular, circular, oval and the like, for providing a channel for necrotic substances to enter the rotary cutting head 20.

The operating handle 30 is provided with a negative pressure suction port 31. One end, away from the rotary cutting head 20, of the dirt suction chamber 13 communicates with the negative pressure suction port 31, and a suction bottle 41 and an aspirator 42 can generate a negative pressure in the dirt suction chamber 13 through the negative pressure suction port 31. When the diseased tissue is cut off by the rotary cutter head 22, the dirt suction chamber 13 can directly suck out the cut diseased tissue to prevent the dirt suction chamber 13 from being blocked.

The rotary cutter head 22 may be rotatably arranged on the housing 21 around its own axis with respect to the housing 21.

Preferably, the rotary cutter head 22 may be a double-leaf jagged blade, so as to cut the diseased tissue better.

Further, in this embodiment, in order to make the rotary cutter head 22 rotatably arranged on the housing 21, an annular groove (not shown in figure) can be arranged on an inner side wall of the housing 21, and a portion of the rotary cutter head 22 can rotatably extend into the groove, so as to complete the fixation of the rotary cutter head 22 and to provide guidance for the rotation of the rotary cutter head 22.

A connecting assembly 24 is arranged at one end, facing the inner tube 11, of the housing 21, and the connecting assembly 24 may be connected with an end portion of the inner tube 11 in a threaded manner, so as to facilitate the replacement of the rotary cutting head 20.

The rotary cutting head 20 further includes a rotating shaft 25 (shown in FIG. 8). One end of the rotating shaft 25 is connected with the rotary cutter head 22, the other end of the rotating shaft 25 is connected with a driving component, such as a motor, in the operating handle 30, so as to control a rotating direction and a rotating speed of the rotary cutter head 22 by the operating handle 30.

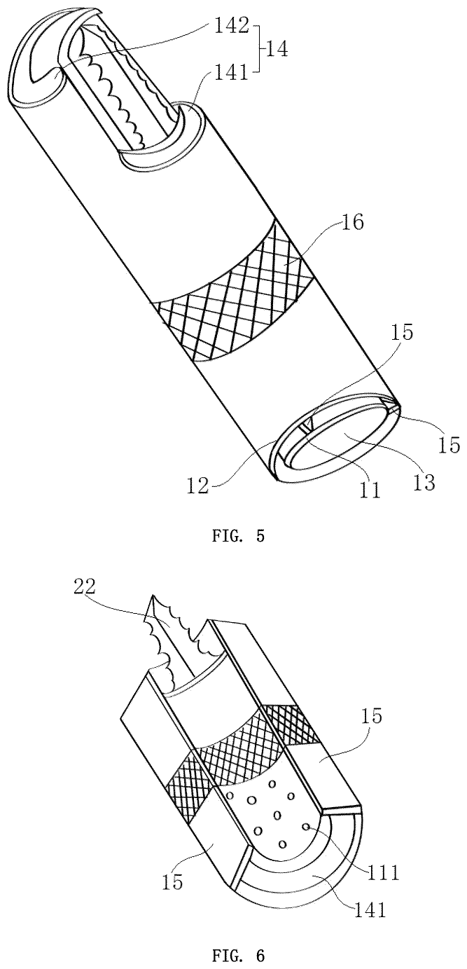

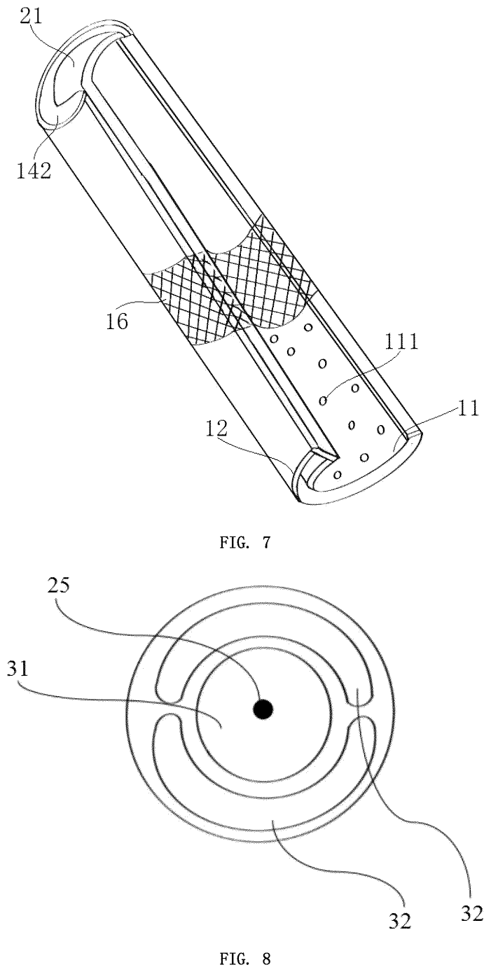

FIG. 5 is a structural schematic diagram after the rotary cutting head and the insertion tube in FIG. 1 are combined. FIG. 6 is a structural schematic diagram of a first irrigation channel in FIG. 5. FIG. 7 is a structural schematic diagram of a second irrigation channel in FIG. 5. FIG. 8 is a schematic diagram of a sectional structure at a joint of the operating handle and the insertion tube. Please continuing to refer to FIG. 1, FIG. 5 to FIG. 8, in this embodiment, partitions 15 are further arranged between the inner tube 11 and the outer tube 12 to divide the irrigation chamber 14 into a first irrigation channel 141 and a second irrigation channel 142. An opening of one end, away from the operating handle 30, of the first irrigation channel 141 is formed in one end, adjacent to the insertion tube 10, of the rotary cutting head 20, and is located on the housing at a position where the rotary cutting opening 23 is located on the housing 21, so as to irrigate the diseased tissue and pus blocked at the rotary cutting opening 23. One end, away from the operating handle 30, of the outer tube 12 is not closed with respect to the housing 21, the second irrigation channel 142 is formed between the outer tube 12 and a side wall of the housing 21 at the rotary cutting head 20, i.e., formed at an entity portion without the rotary cutting opening 23 between the outer tube 12 and the housing 21. An opening of the second irrigation channel 142 is formed in an end portion of one end, away from the operating handle 30, of the housing 21, so as to irrigate the diseased tissue and pus in the body of a patient. The two irrigation channels are provided to provide irrigation solutions with different flow rates and pressures for different regions.

Further, two end portions of the outer tube 12 are respectively flush with end portions of the housing 21 where the rotary cutting opening 23 is not provided, that is, an opening ring of the second irrigation channel 142 is arranged at an end portion, without the rotary cutting opening 23, of the housing 21, so as to facilitate the insertion of the insertion tube 10 and the irrigation to the diseased tissue and the surrounding sites of the human body.

Further, in a long axis direction of the insertion tube 10, positions of the partitions 15 are flush with an end face at an opening of the rotary cutting opening 23, and the partitions 15 extend along the long axis direction of the insertion tube 10.

The operating handle 30 is provided with an irrigation interface 32, which communicates with a pump body 43 and is connected with the first irrigation channel 141 and the second irrigation channel 142. Through the irrigation interface 32, the pump body 43 can inject irrigation solution, such as normal saline, into the first irrigation channel 141 and the second irrigation channel 142 respectively to irrigate the rotary cutting head 20.

Further, the inner tube 11 of the insertion tube 10 is provided with multiple connecting holes 111 for communicating the dirt suction chamber 13 with the irrigation chamber 14, and a flow direction control structure is arranged at the connecting holes 111, such as a check valve or a one-way membrane, to allow one-way flow of the irrigation solution from the irrigation chamber 14 into the dirt suction chamber 13 to irrigate the human tissue in the dirt suction chamber, thus preventing the human tissue from blocking the dirt suction chamber 13.

Further, in this embodiment, the insertion tube 10 may be cylindrical as a whole, so as to facilitate the insertion.

Further, the insertion tube 10 is provided with a bent part 16 and a guide wire (not shown in figure) connected with the bent part 16. The bent part 16 may be made of an elastic material, and is arranged at a position, adjacent to the rotary cutting head 20, of the insertion tube 10. The guide wire may be arranged in the irrigation chamber 14, or directly embedded in a duct inside the outer tube 11, and is connected with a guide wire control structure on the operating handle 30. A length of the guide wire is controlled through the operating handle 30, so as to control the degree of bending of the insertion tube 10.

Further, the guide wire control structure includes a rotating gear, the guide wire is wound on the rotating gear, and a fixing structure for fixing a rotating angle of the rotating gear is arranged on the rotating gear. The guide wire can be tightened or loosened by the rotating gear, so as to adjust the degree of bending of the bent part 16 to adapt to the position of the human diseased tissue. When the rotary cutting head 20 is adjusted to a proper position, a rotating angle of the rotating gear is locked by the fixing structure for debridement operation.

Alternatively, the fixing structure may be a buckle structure, a magnetic attraction structure and the like.

Please referring to FIG. 1 continuously, the operating handle 30 is further provided with a rotating speed regulation button 351, an irrigation water pressure regulation button 353 and a bending regulation knob 354, so as to regulate the parameters of the of the debridement device, such as the rotating direction and speed of the rotary cutter head 22, the negative pressure in the dirt suction chamber 13, the flow rate and pressure of the irrigation solution in the irrigation chamber 14, and the degree of bending of the insertion tube 10.

When performing debridement surgery on diseased tissue through the debridement device for human diseased tissue provided by the present disclosure, the insertion tube 10 can be extended into the human body first, and the degree of bending of the insertion tube 10 can be regulated to make the diseased tissue in contact with a position of the rotary cutting opening 23. Then, the rotary cutter head 22 is controlled to cut the diseased tissue; and meanwhile, the cut diseased tissue is sucked out through the dirt suction chamber 13, and the rotary cutting opening 23 and surrounding human tissue are irrigated through the irrigation chamber 14. After debridement, the insertion tube 10 is removed to complete the operation.

The above is only the preferred embodiment of the present disclosure, rather than limiting in any form. Although the present disclosure has been shown in the preferred embodiment, it is not intended to limit the present disclosure. A person skilled in the art can make some equivalent variations, alterations or modifications to the above technical content without departing from the scope of the technical solutions of the present disclosure to obtain equivalent embodiments. Any simple alteration, equivalent change or modification made to the above embodiments according to the technical essence of the present disclosure without departing from the content of the technical solutions of the present disclosure shall fall within the scope of the technical solutions of the present disclosure.

What is claimed is:

1. A debridement device for human diseased tissue, comprising an insertion tube, a rotary cutting head and an operating handle, wherein the insertion tube comprises an inner tube and an outer tube sleeved outside the inner tube, a dirt suction chamber is formed in the inner tube, and an irrigation chamber is formed between the inner tube and the outer tube; the rotary cutting head comprises a housing and a rotary cutter head arranged in the housing; an interior of the housing communicates with the dirt suction chamber, a rotary cutting opening for exposing the rotary cutter head is formed in the housing, and a notch corresponding to the rotary cutting opening is formed in the outer tube; the operating handle is connected with the insertion tube and the rotary cutting head to control the rotary cutter head to cut human tissue at the rotary cutting opening, to suck dirt from the dirt suction chamber, and to irrigate the rotary cutting head through the irrigation chamber.

2. The debridement device for human diseased tissue according to claim 1, wherein the rotary cutting opening is laterally formed in the housing, and a top of the housing is a blind end and is arc-shaped.

3. The debridement device for human diseased tissue according to claim 1, wherein the operating handle is provided with a negative pressure suction port;
one end, away from the rotary cutting head, of the dirt suction chamber communicates with the negative pressure suction port, and a suction bottle and an aspirator form a negative pressure in the dirt suction chamber through the negative pressure suction port.

4. The debridement device for human diseased tissue according to claim 1, wherein the rotary cutter head is rotatably arranged in the housing about an axis of the rotary cutter head.

5. The debridement device for human diseased tissue according to claim 4, wherein the rotary cutting head comprises a rotating shaft, one end of the rotating shaft is connected with the rotary cutter head, and an other end of the rotating shaft is connected with a driving component in the operating handle, and the driving component drives the rotary cutter head to rotate through the rotating shaft.

6. The debridement device for human diseased tissue according to claim 1, wherein partitions are arranged between the inner tube and the outer tube to divide the irrigation chamber into a first irrigation channel and a second irrigation channel; an opening of one end, away from the operating handle, of the first irrigation channel is formed in one end, adjacent to the insertion tube, of the rotary cutting head, and is located on the housing at a position where the rotary cutting opening is located on the housing; one end, away from the operating handle, of the outer tube is not closed with respect to the housing, and the second irrigation channel is formed between the outer tube and a side wall of the housing at the rotary cutting head; and an opening of the second irrigation channel is formed in an end portion of one end, away from the operating handle, of the housing.

7. The debridement device for human diseased tissue according to claim 6, wherein two end portions of the outer tube are respectively flush with end portions of the housing where the rotary cutting opening is not provided.

8. The debridement device for human diseased tissue according to claim 6, wherein the inner tube is provided with a plurality of connecting holes for communicating the dirt suction chamber with the irrigation chamber, and a flow direction control structure is arranged at the connecting holes to allow one-way flow of an irrigation solution from the irrigation chamber into the dirt suction chamber.

9. The debridement device for human diseased tissue according to claim 1, wherein the insertion tube is provided with a bent part and a guide wire connected with the bent part, the bent part is arranged at a position, adjacent to the rotary cutting head, of the insertion tube, the bent part is connected with a guide wire control structure on the operating handle through the guide wire, and a length of the guide wire is controlled through the operating handle to control a degree of bending of the bent part.

10. The debridement device for human diseased tissue according to claim 9, wherein the guide wire control structure comprises a rotating gear, the guide wire is wound on the rotating gear, and a fixing structure for fixing a rotating angle of the rotating gear is arranged on the rotating gear.
